(19) United States
(12) Patent Application Publication
(10) Pub. No.: US 2005/0228219 A1
(43) Pub. Date: Oct. 13, 2005

(54) Dual-motion auto-stimulation device

(76) Inventor: Stephen W. Jewell, League City, TX (US)

Correspondence Address: KUFFNER & ASSOCIATES, 2476 BOLSOVER ST. #534, HOUSTON, TX, 77005, US

(21) Appl. No.: 11/145,335

(22) Filed: Jun. 3, 2005

Related U.S. Application Data
(62) Division of application No. 10/748,818, filed on Dec. 30, 2003, now Pat. No. 6,902,525.

(51) Int. Cl. A61F 5/00

(52) U.S. Cl. 600/38

(57) ABSTRACT

A device designed to take rotary power from an external power source such as a cordless electric screwdriver and transmit that energy into both rotary and reciprocating linear activation of sexual devices used by both males and females for the purpose of masturbation and auto-stimulation is disclosed. The combination of rotational stimulation with reciprocating linear stimulation is apparently remarkably satisfying despite the absence of such stimulation during normal person-on-person sexual contact. The device is simple, light weight, small, powerful, modestly priced, handheld or hands-free, user serviceable, hygienic, gender neutral, sexual orientation neutral, convenient, rugged, multi-motion, safe, reliable, quiet, fully submersible, positionally versatile and sexually satisfying.

### REFERENCE TO PRIOR RELATED APPLICATION

This application is a division of co-pending prior application Ser. No. 10/748,818, filed Dec. 30, 2003, to be issued as U.S. Pat. No. 6,902,525 on Jun. 7, 2005.

### BACKGROUND OF THE INVENTION

1. Field of the Invention

The present invention relates to the field of sexual stimulation devices used by both males and females for the purpose of sexual stimulation. In particular, the invention relates to mechanical devices adapted to transmit rotary energy into both rotary and reciprocating linear activation for the purpose of masturbation and/or stimulation of one's partner during sexual play.

Various known devices for stimulating the female genitalia have been developed for the purpose of providing sexual gratification and climax during sexual intercourse, foreplay, and masturbation. Generally, they are used for personal pleasure and recreation by providing the user healthy sexual outlets. They are sometimes used in combination with sex-therapy programs. The sexual devices can provide the user an opportunity for sexual exploration and development, alleviate fears and anxiety about sexual performance, aid in the development of one's sensual capacities, and even facilitate verbal, emotional and sexual communication between sex partners. The most common form of such device is the vibrator, which operates by applying pressure, friction, and vibration to the clitoral region, walls of the vagina and/or external areas of the genitalia.

There are far fewer devices available for male auto-stimulation. These are principally soft elastomer sleeves into which the penis is inserted and manually massaged.

2. Description of the Background Art

The demand for products used for sexual self-satisfaction and/or to increase satisfaction between couples, be they heterosexual or homosexual can be illustrated by the plethora of product offerings filling sex shops and adult stores, and orderable online from literally thousands of Internet retailers. Estimates indicate that the annual sales for so-called “sex toys” surpasses a billion U.S. dollars. Clearly, there is a large demand, and a large supply, of devices for use in sexual stimulation.

Of course, not all sex products for sale are devices. Many are lotions and lubricants, condoms, lingerie, and other sexual paraphernalia. Of those that can be considered auto-erotic stimulating devices, many or most are hand held and or operated. These include silicon and other soft plastic elastomer sleeves for penile stimulation and many non-operating penis substitutes intended for vaginal and rectal insertion. Those that actually provide some form of mechanical stimulation usually do so with small internal motors generating predominantly vibratory action operating on two, three or four AA or AAA batteries. Like their un-powered cousins, the user is expected to impart most of the intercourse simulating motion.

There are a few dildos that claim linear thrusting in the head of the device but, to the extent these claims are true, this action is not a close intercourse simulation.

For both men and women, what is required to simulate the act of copulation involves rather long strokes of the entire device that terminate near full insertion. This is not to suggest that other forms of stimulation, such as vibration and rotation, are not also appropriate for auto-stimulation, but simply to point out that they have not routinely been a part of one-on-one human sexual contact over our evolutionary history.

These mass-marketed devices retail from as little as ten dollars for a simple small vibrator up into the sixty to eighty dollar range for more sophisticated devices. They are almost without exception less than $100 in America. Overall, they may be characterized as cheap, fragile, underpowered, gender specific, single action, often unhygienic when badly insulated internal electrical components prevent proper washing by means of submersion, unserviceable by the user, and disappointingly unsatisfying sexually. No sexual partner need worry about losing their mate's affection to any of these machines.

The background paragraph of U.S. Pat. No. 6,142,929, issued to Padgett in November of 2000, states, “There exists a need for a cheap, effective, safe, private, disease free, non impregnating, and natural like way for a woman to achieve sexual gratification with a sexual stimulation apparatus.” While this may be correct, the statement's applicability ought to be extended to the males of our species as well.

There are two sexual stimulation currently being sold that reportedly are very popular. The Sybian is a machine designed for females, in the use of which the woman sits atop and is penetrated by a mechanically actuated dildo. Two motors provide differing action; one vibration and a second a peculiar form of rotation. The dildo doesn't actually revolve within the user but the top sweeps through a circular arc while the base remains stationary. This is one of the few dual-action machines presently available.

A second product is sold for male users. It is called the Venus and is a patented device (U.S. Pat. No. 5,501,650 issued to Gellert in March 1996). This device is a pneumatically operated penile sleeve. A reciprocating pump causes the air pressure on the outside of the sleeve to rise and fall so that the sleeve moves up and down on the penis of the user. The Venus 2000 receives glowing reviews, but is once again single action, and is of course gender specific.

Inventive minds have been hard at work building machines that provide sexual release. Nevertheless, we characterize this second collection of low volume custom built machines as large, heavy, cumbersome, expensive, powerful, often noisy, gender specific, generally unserviceable by unskilled user, single action, unhygienic, and in many cases plainly dangerous.

A third class of machines are those that were not sexual stimulation devices by design, but are instead machines built for other purposes and put to what can be termed novel use. One such machine is simply a reciprocating saw found in many home workshops to which a dildo is attached. These machines typically provide about two inches or less of stroke. In another innovative twist, a dildo attachment device with a ¼ inch hex drive on the back enables direct attachment to an electric drill or other rotary energy source, thereby causing the dildo to rotate inside the male or female orifice of preference. Any linear stroke must be manually provided by the user.

The attachment mechanism mentioned above is not unique to this device. There exists an industry standard adapter connection called the Vac-U-Loc® that fits an entire line of dildos offered by Health Devices Corp. (doing business as Doc Johnson), one of the largest purveyors of sexual devices. The fact that this prominent manufacturer maintains this line of products, which are functional only when connected to another device, suggests that there are a large number of various sexual devices to which these stimulation devices might be attached.

Some items in this field do provide rotational stimulation, which users appear to find satisfying despite the absence of such stimulation during normal person-on-person sexual contact over our evolutionary development. In fact this type of stimulation is difficult to attain between two sexual partners. Once again, inventive minds have worked on this articulation problem. The so-called “sex swing” can be either supported from the ceiling or some other overhead structure or may be purchased with purpose-designed and marketed freestanding frames. They provide for one sexual partner to be suspended in slings of various designs attached to the overhead structure by springs or bungee cords.

While there are many sexual positions suggested as effective for use with these swings, there is only one practical one in which rotational stimulation (in-conjunction with linear thrust afforded by the springs or bungee) is possible. This of course is with one partner suspended directly above the other where the suspended partner can be freely rotated. The swings themselves may be relatively modestly priced, but they are difficult to set up, get suspended in, and put away out of the children and neighbors' sight. There is the added danger in the aforementioned configuration that an equipment failure, ceiling or ceiling attachment failure, or a simple slip by the suspended partner within the sling might end the session with catastrophic consequences. Swings are an extreme measure to resort to in order to experience simultaneous insertion and rotation.

For all these reasons there exists a need for a device that provides both rotary and linear reciprocating activation of sexual devices used by both males and females for the purpose of sexual stimulation. Such a device would be desirable only if it could be convenient, simple and safe, easy to handle, light and small, yet powerful and still be modestly priced, rugged, reliable and satisfying.

### SUMMARY OF FIGURES

FIG. 1 shows a perspective view of the assembled device of the preferred embodiment in its female configuration. The dotted lines are added to show how the female attachment is connected to the oscillating drive train shuttle within the protective housing.

FIG. 2 shows a perspective view of an embodiment of the invention with the cover 9 of the gearbox exploded outwardly to reveal the gear train power transmission working components.

FIG. 3 is a rotated right side view of the device shown in FIG. 2.

FIG. 4 is a cross-sectional view of the device shown in FIG. 2 as viewed along the line 4-4 in FIG. 3.

FIG. 5 is a reverse perspective view the device shown in FIG. 2 with the stand-off housing and other outer housing components removed so as to illustrate the power transmission and dual-motion aspects of the invention.

FIG. 6 illustrates in two different views the detail of a preferred form of shuttle cam follower groove profiles.

FIG. 7 shows a perspective view of the detail of a preferred form of stand-off assembly with the housing 20 removed.

FIG. 8 illustrates the expanded stand-off assembly of FIG. 7 configured to carry a male masturbation device (not shown) and having the protective housing in place.

### SUMMARY OF THE INVENTION

The present invention provides a light weight, portable, multi-motion device that provides all of the above characteristics and, in addition, is adapted for handheld or hands-free operation, thereby enhancing sexual activity and providing satisfying sexual stimulation. The device may be fabricated in a cost effective and simple manner and thus will be relatively inexpensive while still being rugged, reliable, hygienic, quiet, fully submersible and positionally versatile. The device is simple and easy to use and operate requiring little instruction.

It is a feature of the present invention that a device made according to the invention is capable of being adapted to provide mechanical stimulation to the genitalia of a woman or a man and thus is gender and sexual orientation neutral.

It is also a feature of the present invention that a device made according to the invention can take rotary power and transmit that energy into rotary and linear reciprocating activation of sexual devices used by both males and females for the purpose of sexual stimulation. In the embodiment most thoroughly described below, rotary power is provided from an external power source such as a cordless electric screwdriver or drill.

The invention is embodied in a portable, lightweight, hand held apparatus, adapted to be used by both males and females, for operative coupling with a suitable sexual stimulation device and providing a unique combination of simultaneous rotating and linearly reciprocating motion.

6. The device of claim 1, wherein the connecting means includes a universal adapter suitable to receive and attach to a variety of sexual stimulation devices.

7. The device of claim 6, wherein the connecting means includes an easy off-easy on attachment.

In this embodiment, the invention comprises first a connecting means for operatively coupling with a suitable sexual self stimulation device, adapted for either male or female use. Associated with the connecting means is any suitable power mechanism for providing rotating motion through the connecting means to the stimulation device. Also associated with the connecting means and the rotatingly motivating means is any suitable power mechanism for providing linear back and forth (i.e., reciprocating) motion to the stimulation device at the same time as it is rotatingly motivated, thereby producing a simultaneous reciprocating and rotating motion of the stimulation device.

An optional, but important, element of the invention is a stand-off assembly, adapted to keep the operating components of the power transmission assembly and the stimulation device safely away from the user. In its preferred embodiment, the stand-off assembly comprises a tube adapted to be secured in place at the distal end of the connecting means.

The power transmission assembly represents the key to the unique motion of the present invention. In a preferred embodiment, it comprises a linear actuation assembly adapted to receive rotary motion from the drive shaft; and rotary energy transmission means associated with the drive shaft to transmit rotary energy from the drive shaft to the linear actuation assembly.

The linear actuation assembly comprises a generally tube-shaped outer protective transmission housing, a transmission fork within the housing that is fixedly attached to the drive shaft; and a cammed shuttle connected to the transmission fork and adapted to receive rotational energy therefrom. The shuttle is adapted to fit within the transmission housing and to move linearly therein. A cam follower is fixedly mounted to engage the cammed shuttle and induce linear reciprocation of the shuttle within a confined space as it simultaneously rotates.

The shuttle, in its preferred form, has at least one suitably configured cam groove cut into the outer surface thereof. A corresponding cam follower is mounted on the transmission housing and adapted to engage the cam groove in the shuttle and thereby induce linear reciprocation of the shuttle following the profile of the groove when the shuttle rotates.

A rotatable transmission fork is slidably located within the linear shuttle and fixed in place laterally relative to the transmission assembly housing. It is attached to and receives rotational motion from an appropriate rotary energy transmission means, such as a gear box assembly associated with the power connection and the drive shaft. The transmission fork is designed and adapted such that rotation of the laterally fixed transmission fork is transmitted to the shuttle, but the shuttle is still free to move linearly within the transmission housing.

The attachments assembly, affixed to the distal end of the power transmission assembly so as to move in synchrony with the active components of the power transmission assembly, is made to connect with and transmit motion of the device to a suitably selected stimulation mechanism. In its preferred form, the attachments assembly includes a universal adapter, suitable to receive and attach to a multiplicity of self stimulation devices.

A stand-off assembly, adapted to keep the operating components of the power transmission assembly and the stimulation device safely away from the user, is included with the preferred embodiment of the apparatus of this invention. The preferred form of such stand-off assembly includes a tube adapted to be secured in place on the distal end of the power transmission assembly and fit over the selected stimulation mechanism, be it male or female, to protect the user from injury.

### DETAILED DESCRIPTION OF THE PREFERRED EMBODIMENT

The device of the present invention is designed to take rotary power from an external power source, such as a cordless electric screwdriver or the like, and transmit that energy into both rotary and reciprocating linear activation of sexual devices used by both males and females for the purpose of masturbation.

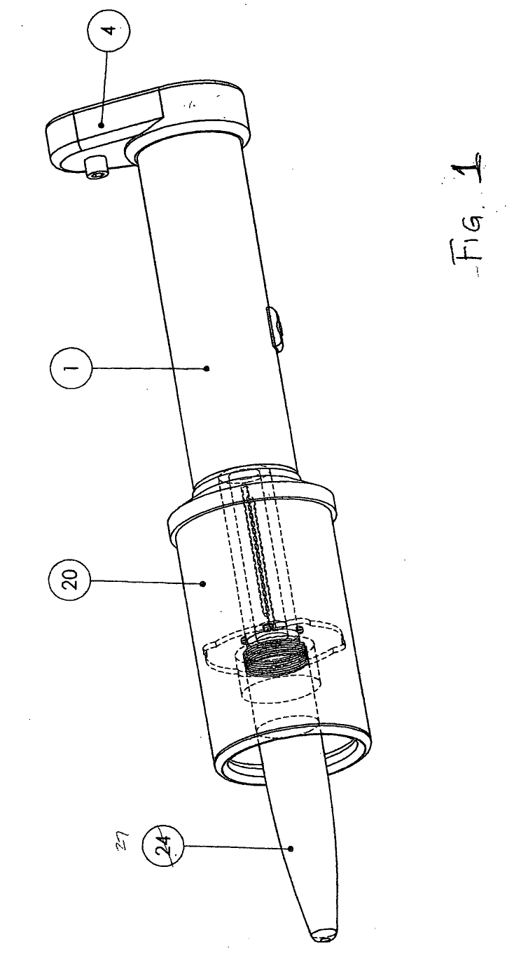

FIG. 1 shows the assembled device with the attachments assembly configured for use by females. The female stimulation device 27 is shown attached to the device of the invention but is not part of the invention. The attachments assembly for the male configuration is longer and includes a few additional pieces discussed further below.

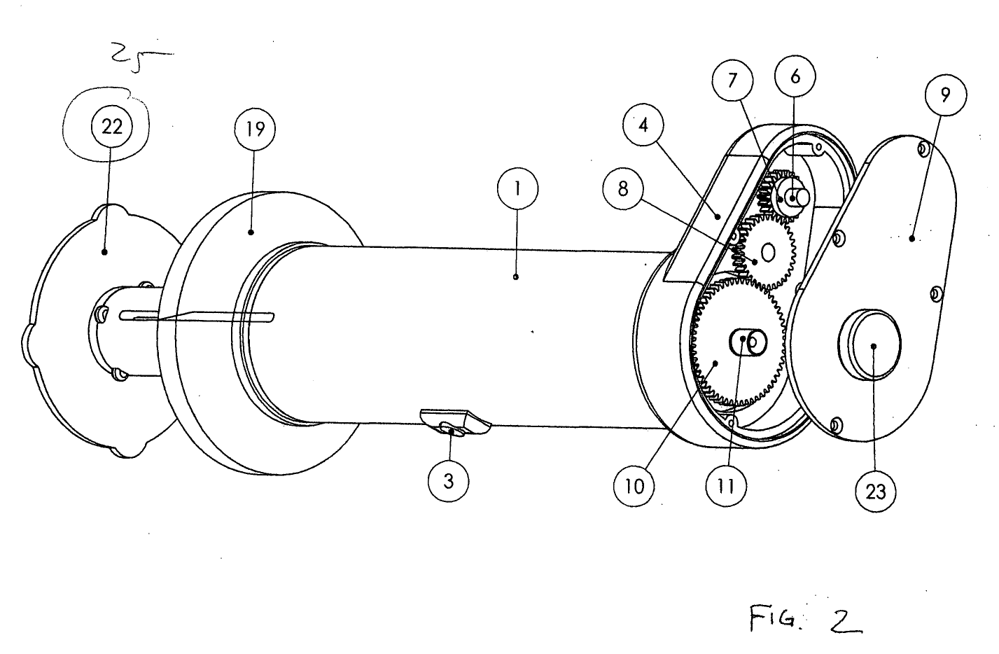

With reference to FIGS. 1 and 2, the power transmission assembly 1 comprises the key aspect of the invention and contains the majority of its parts. In its preferred form, it is comprised of two major subassemblies; a gearbox assembly 100 and a linear actuation assembly 200. The gearbox housing 4 is rigidly attached to the transmission housing 18 and together they maintain all their respective components in their proper working relationship. FIG. 2 shows the power transmission assembly 1 in its entirety with the gearbox cover 9 exploded to reveal the gear train. All these items are suitably fixed in place in working association with each other. The drive gear 7 is attached securely to the drive shaft 6, which is driven by the external power source. It in turn drives an idler gear 8, which revolves freely on a shaft molded into the inside of the gearbox. The idler gear 8 transmits the rotation to the driven gear 10. The driven gear otherwise turns freely on the driven gear shaft 11. A capture screw 26 secures the driven gear shaft through a suitable opening in the cover 9.

It will be obvious to anyone familiar with the art that other mechanisms could be employed to transmit the rotary energy from the drive shaft to the drive train mechanism contained inside the transmission housing 18. Belts and pulleys in a number of forms, frictional wheels, or even chain would all be effective for this purpose. However, the disclosed embodiment is presently the preferred one.

The transmission housing 18 is adapted to be secured at its distal end to the stand-off assembly 300. In the embodiment shown in the drawings (FIG. 1), this is done by means of a funnel connector 19, which is adapted at one end to attach to the transmission housing 18 and at its distal end to connect with the stand-off housing 20.

In the embodiment shown in the Figures, the drive shaft 6 and driven gear shaft 11 are both stainless steel and the gears and most other parts are molded plastic unless noted differently. Obviously, the choice of materials for all these components is large and varied.

The drive shaft 6 passes through the underside of the gearbox housing where it engages the drive gear as already discussed. As will be observed, the underside of the gearbox cover 9 is adapted to provide receiving holes for the metal shafts and, when held in place atop the gearbox housing 4 by the four screws (not shown), maintains stability in the shafts and close tolerances atop the gears (7,8 and 10 in FIG. 2) to keep them in place on their respective shafts.

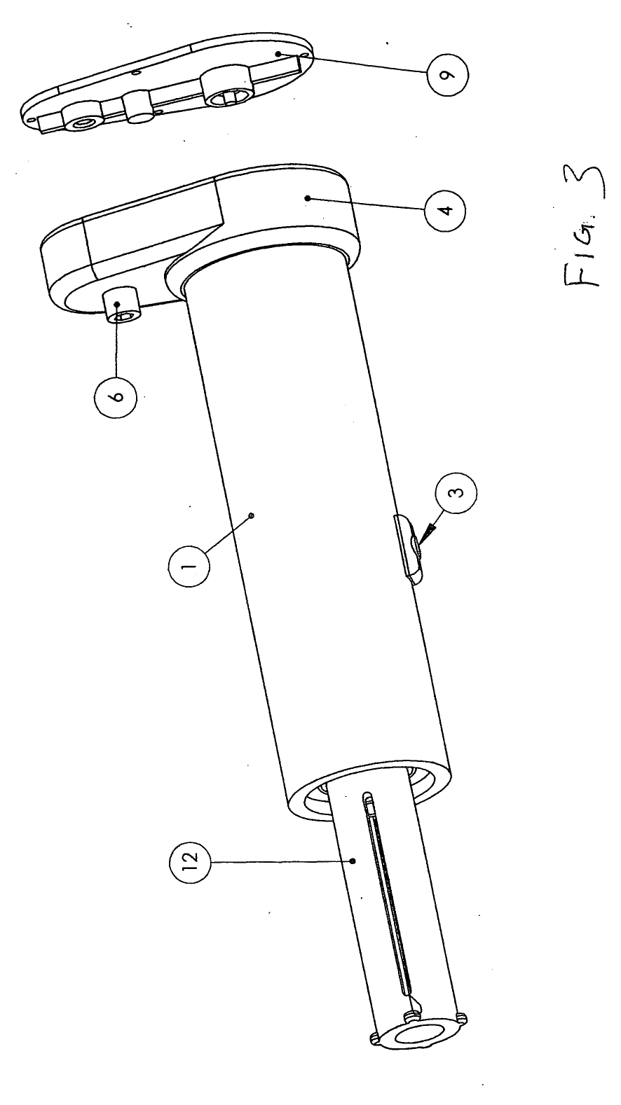

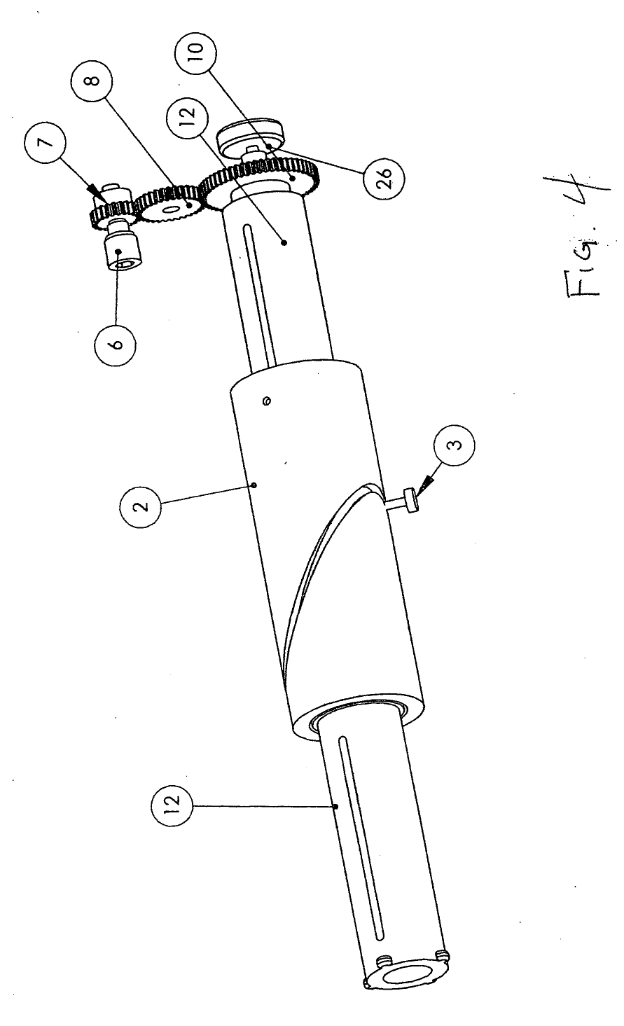

FIG. 3 is the rotated right side view of the power transmission assembly 100 and indicates the section line that gives rise to FIG. 4. FIG. 4 reveals the interaction between the gears and the remaining drive train components contained within the transmission housing 18. The driven gear 10 is affixed securely atop a transmission fork 12 and imparts rotational energy into it. Together the transmission fork 12 and the driven gear 10 are fixed in place laterally relative to the transmission housing 18 and the gearbox housing 4 by the driven gear shaft 11 and the capture screw 26. Also shown in FIG. 4 is the shuttle 2, which is the active component responsible for generating the linear motion.

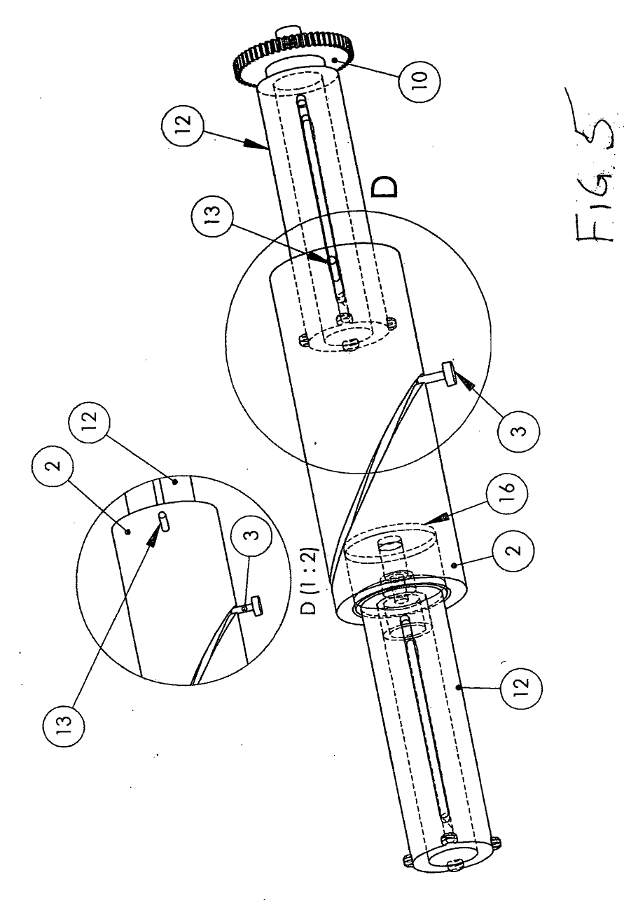

With reference to FIG. 5, it will be seen that the transmission fork 12 is slotted completely through along a portion of its length. The transmission fork 12 fits loosely and slidably inside the shuttle 2. A drive pin 13 is secured in two opposing holes along the centerline of the shuttle and passes through the slot in the fork 12 leaving the drive pin 13 free to move linearly within the slot in fork 12.

One can see then that, when the transmission fork is inserted into the shuttle 2 and the drive pin inserted through the holes in the shuttle and extending through the slot in the transmission fork, rotation of the laterally fixed transmission fork 12 is transmitted to the shuttle 2, but the shuttle is free to move linearly within the transmission housing 18. Depending on the choice of materials and their frictional and wear characteristics, it is possible that sleeve bearings (not shown) may be necessary for the inner surface to roll freely on the drive pin 13 and the outer surface to roll against the slot in the transmission fork 12.

The shuttle 2 is what imparts the linear motion to later components in the drive train. Cut into the outside surface of the shuttle, is a groove as indicated in FIG. 5. A cam follower 3 is mounted on the transmission housing 18 and is engaged inside the groove profile in the shuttle 2. Because the cam follower 3 is fixed linearly with regards to the transmission housing 18 (FIG. 1-5), rotation of the shuttle 2 induced by the drive pin 13 then causes the shuttle 2 to move linearly within the transmission housing 18. The shuttle 2 when rotated will reciprocate linearly following the profile of the groove on the shuttle.

We note at this point that by removing the capture screw 26 and loosening the cam follower 3 so it no longer engages the groove in the shuttle 2, that the entire drive mechanism will slide free of the transmission housing 18. This provides for easy disassembly and cleaning of the machine. In fact, because the components are all stainless steel and plastic the device can be disassembled and placed in the dishwasher.

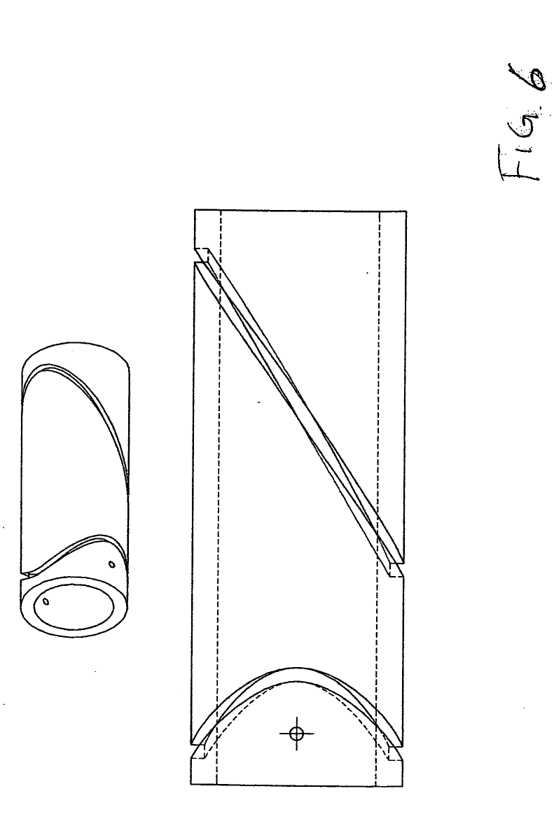

FIG. 6 shows the detail of a preferred form of shuttle. As indicated, a shuttle 2 is not limited to a single or simple groove profile. There could be multiple grooves following different paths around the shuttle causing different linear motion to be imparted. In fact a single circular groove would cause the shuttle to remain in a fixed linear position. Shown is a shuttle with two different profiles. To make use of multiple profiles on a single shuttle would require multiple holes in the transmission housing 18 capable of accepting the cam follower 3 so as not to exceed the freedom of motion afforded by the slots in the transmission fork 12. In one embodiment of this configuration, a flashlight-like sliding switch is mounted on the side of the transmission housing 18 and causes different cam followers to engage different slots while the machine is in motion, thereby allowing the user to quickly alternate between different linear actions. And of course the shuttle itself is easily removable. Thus, during a single use of a device according to the present invention, a user could interchange multiple shuttles having a variety of movement profiles.

Having brought up the possibility of complicated profiles on the shuttle, we should also point out the same is possible for the rotary motion. Remember the rotary motion delivered to the transmission fork 12 is provided externally, but the rotary motion imparted to the shuttle 2 could be transformed by altering the shape of the slots in the transmission fork in which the drive pin 13 travels. Were these slots made to trace a sinusoidal path, or the like, along their length, a rotational velocity oscillation, or “wobble”, would result.

Returning to FIG. 4, we can see the mechanism by which the rotary and linear action of the shuttle 2 is carried down the drive train. A transmission extension 23-is affixed inside the other end of the shuttle by way of a clutch 15 and clutch washer 16. The clutch assembly is a very important safety component in the system protecting both the machine and the user. The clutch 15 is made of a pliable elastomer that when compressed by the clutch washer 16 will expand radially outwards to create frictional contact with the inside of the shuttle 2. The amount of friction is adjusted by means of the clutch screw 17 that threads into the clutch base 14. This in turn is attached rigidly to the top of the transmission extension 23. The adjustment is actually accomplished with a long screwdriver, which engages a slot (not shown) in the bottom of the clutch screw 17. Together these components (14-17) form the clutch assembly 30 whose position is indicated in hidden lines on FIG. 5.

If for any reason the transmission extension 23 or any down stream components affixed to it were prevented from moving while the machine was activated, the clutch would release to prevent damage to the machine or injury to the user. The release is effective in all directions and dimensions of motion. In the event the shuttle 2 was traveling down towards to user, and the business end were prevented from doing so, the clutch could release and slide up inside the shuttle 2 until encountering the fixed transmission fork 12 at the other end which is equivalent to the fully retracted state of the machine.

In the alternate case, where the shuttle is retracting and moving away from the user, if any problem caused the downstream mechanisms to resist following it, the clutch will release and simply pull free from the shuttle. And lastly, in the event of two much rotary resistance, the clutch will release rotationally.

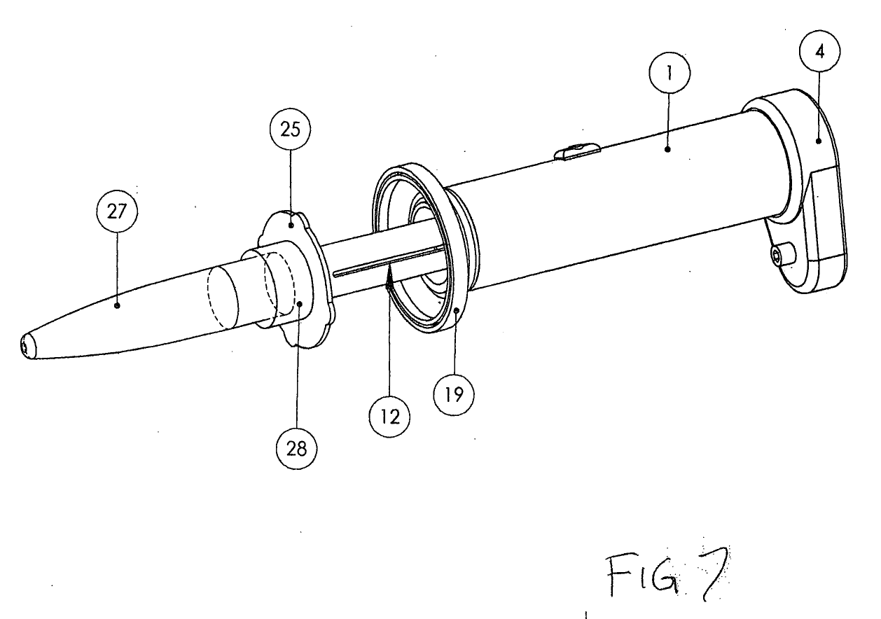

The device shown in FIG. 7 is much like that in FIG. 1 with the stand-off housing 20 removed and configured with a female stimulator 28 (not part of the invention). The stand-off assembly 300 serves to keep the operating components of the machine safely away from the user. Inside the stand-off assembly 300, a universal adapter 25 is affixed to the distal end of the transmission extension 23 and moves in synchrony with it. It is this piece to which specific devices will be attached to activate various masturbation devices. Obviously, the only requirements of the universal adapter 25 is that it fit securely on the end of the transmission extension 23, as shown in FIG. 7 and be adapted to connect with and transmit its movement to a suitable selected stimulation device. Different specific device adapters 24 to be affixed to the universal adapter 25 are available for both male and female sexual stimulation devices.

While rotational stimulation is satisfying to many users as a novel stimulus, there is nothing natural about rotation in the act of intercourse. The universal adapter 25 is always rotating and following the linear profile (if any) of the shuttle 2. The device adapters 24 can be affixed to the universal adapter 25 by way of a free-turning shaft that allows the final motion imparted to the user to be only linear. In combination then of various shuttle profiles and fixed or free-turning attachment of the device adapters, the user can select any combination of rotation, reciprocation, or both.

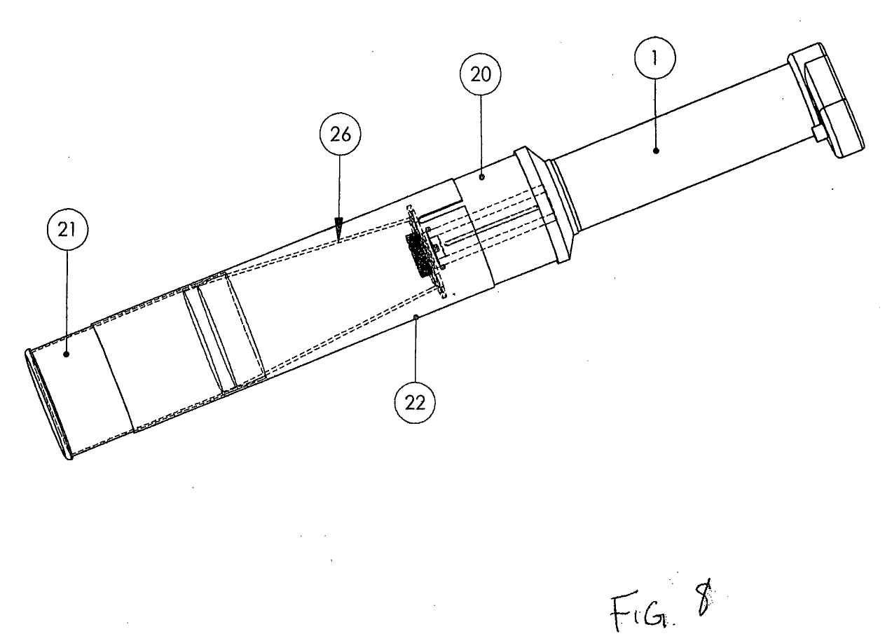

FIG. 8 illustrates the expanded stand-off assembly configured to carry a male masturbation device 28 (not part of the invention). There are two additional components. There is a stand-off extender 22 which slips over the female stand-off housing 20. This enables the user to adjust the depth of the masturbation device within the overall assembly. Once the adjustment is made, the stand-off extender 22 can be set in place snuggly with relation to the female stand-off housing 20 by way of a belt (not shown) that compresses it around the inner stand-off housing 20. Finally, a male safety sleeve 21 slides freely inside the end of stand-off extender 22. The safety sleeve is constructed essentially the same as the female stand-off housing 20, but serves a different purpose. The male masturbation device 28 moves freely inside the male safety sleeve 21, which is held concentric with the female stand-off tube 20 by way of the stand-off extender 22. The safety sleeve 21 protects the male user from being pinched between the male masturbation device 28 and any component of the stand-off assembly 300. Were the user to be pinched here, the male safety sleeve would simply slide out in unison with the masturbation device preventing injury to the user.

The present invention described herein, therefore, is well adapted to carry out the objects and attain the ends and advantages mentioned, as well as others inherent therein. While a presently preferred embodiment of the invention has been described for purposes of disclosure, numerous changes in the details of procedures may be made for accomplishing the desired results. For example, the embodiment illustrated in the drawings derives all forms of motion by strictly mechanical interaction of the shuttle 2, the transmission fork 12, and the cam follower 3. In a different embodiment the action of the shuttle could be electromagnetically induced. The gear train and transmission fork 12 could be eliminated while the rest of the drive train safety mechanisms remain effective. Electromagnetically actuating the shuttle would provide nearly limitless versatility of motion by use of inexpensive internal or external computer programmable controllers, and would allow even greater safety. Feedback from the power source could instantly disable the machine when resistance to motion in any direction caused the current draw to exceed some threshold.

It will also be apparent that the diameter and length of the shuttle and transmission tube could be made large enough such that the shuttle itself could carry a male masturbation sleeve. In such an embodiment the standoff assemblies are not necessary, therefore a shorter device is made possible at the expense of broader and somewhat heavier machine whose operation and activation would otherwise be identical. Further, a collection vessel may be included with such a male-configured device so as to collect semen. In this way, the device of the present invention could be used for the collection of semen in animal breeding programs.

As described above, in the preferred embodiment, the linear reversing actuation is effected by way of the cam follower moving along one or more grooves in the outside surface of the rotating shuttle. It should be evident that the system would work equally well with the groove on the inside of the tube and the cam follower attached to the shuttle. Another alternative for a linear reversing actuator would be a mechanism not unlike the commercially available level-wind systems found on casting type fishing reels (sometimes known as a diamond-reverser).

Modifications of the apparatus, procedures and conditions disclosed herein that will still embody the concept of the improvements described should readily suggest themselves to those skilled in the art, and are intended to be encompassed within the spirit of the invention presently disclosed herein as well as the scope of the appended claims.

1. A device useful for operative coupling with a sexual stimulation device and providing a unique combination of motions comprising:
a connecting means for operatively coupling with said stimulation device;
means associated with the connecting means for rotatingly motivating said stimulation device, and
means, associated with the connecting means and the rotatingly motivating means, for linearly motivating the stimulation device at the same time as it is rotatingly motivated, thereby producing a simultaneous reciprocating and rotating motion of the stimulation device.

2. The device of claim 1 wherein said sexual stimulation device is configured primarily for female use.

3. The device of claim 1 wherein said sexual stimulation device is configured primarily for male use.

4. The device of claim 1, further including a stand-off assembly adapted to safely separate the user from the operating components of the stimulation device.

5. The device of claim 4, wherein said stand-off assembly comprises a tube adapted to be secured in place at the distal end of the connecting means.

6. The device of claim 1, wherein the connecting means includes a universal adapter suitable to receive and attach to a variety of sexual stimulation devices.

7. The device of claim 6, wherein the connecting means includes an easy off-easy on attachment.

8. A lightweight portable apparatus for producing simultaneous rotating and linearly reciprocating motion and transmitting such motion to a suitable self stimulation masturbation device, said apparatus comprising:
a power transmission assembly including a drive shaft;
connection means associated with the drive shaft adapted to enable rotatable connection of the drive shaft to an external rotating power source; and
attachment means affixed to the distal end of said drive shaft and adapted to attach to and transmit motion from the drive shaft to a selected suitable self stimulation device
said power transmission assembly comprising
a linear actuation assembly operatively coupled the drive shaft; said linear actuation assembly comprising
a transmission fork fixedly attached to said drive shaft;
a cylindrical shuttle connected to and co-axial with said transmission fork and adapted to receive rotational energy therefrom, and
rotary energy transmission means associated with said drive shaft to transmit rotary energy from the drive shaft to the linear actuation assembly.

9. The apparatus according to claim 8, wherein the power transmission assembly includes a gearbox assembly containing at least two gears and the linear actuation assembly includes a clutch assembly and a cam and groove assembly.

10. The apparatus according to claim 8, wherein the attachment means includes a universal adapter suitable to receive and attach to a multiplicity of self stimulation devices.
